(19) United States
(12) Patent Application Publication
(10) Pub. No.: US 2019/0185847 A1
(43) Pub. Date: Jun. 20, 2019

(54) Improving a Microorganism by CRISPR-Inhibition

(71) Applicant: Novozymes A/S, Bagsvaerd (DK)

(72) Inventors: Michael Dolberg Rasmussen, Vallensbaek (DK); Poul Erik Pedersen, Farum (DK); Carsten Hjort, Smoerum (DK); Barbara Cherry, Winters, CA (US)

(73) Assignee: Novozymes A/S, Bagsvaerd (DK)

(21) Appl. No.: 16/309,651

(22) PCT Filed: Jul. 5, 2017
(86) PCT No.: PCT/US2017/040689

§ 371 Date: Dec. 13, 2018

Related U.S. Application Data
(60) Provisional application No. 62/358,644, filed on Jul. 6, 2016.

(51) Int. Cl. C12N 15/11; C12N 9/22; C12N 15/75

(52) U.S. Cl. CPC C12N 15/11; C12N 9/22; C12N 2800/80; C12N 2310/20; C12N 15/75

(57) ABSTRACT

The present invention relates to methods for improving at least one property of a microorganism host cell by repressing the expression of one or more genome target sequence of interest by CRISPR-inhibition as well as the resulting host cells and methods of production emplying said host cells.

### REFERENCE TO SEQUENCE LISTING

This application contains a Sequence Listing in computer readable form, which is incorporated herein by reference.

### FIELD OF THE INVENTION

The present invention relates to methods for improving at least one property of a microorganism host cell by repressing the expression of one or more genome target sequence of interest by CRISPR-inhibition as well as the resulting host cells and methods of production emplying said host cells.

### BACKGROUND OF THE INVENTION

The so-called CRISPR (clustered regularly interspaced short palindromic repeats) Cas9 genome editing system originally isolated from S. pyogenes has been widely used as a tool to modify the genomes of a number of eukaryotes. However, only a few publications have reported the use of this editing system in bacteria.

The Cas9 enzyme has two RNA-guided DNA endonuclease domains capable of targeting specific genomic sequences. The system has been described extensively for editing genomes in a variety of eukaryotes (Doudna and Charpentier, 2014, Genome editing. The new frontier of genome engineering with CRISPR-Cas9, Science 346(6213): 1258096), E. coli (Jiang et al., 2013, RNA-guided editing of bacterial genomes using CRISPR-Cas systems, Nat. Biotechnol. 31(3): 233-9), yeast (DiCarlo et al., 2013, Genome engineering in Saccharomyces cerevisiae using CRISPR-Cas systems, Nucleic Acids Res. 41(7): 4336-4343), Lactobacillus (Oh and van Pijkeren, 2014, CRISPR-Cas9-assisted recombineering in Lactobacillus reuteri. Nucleic Acids Res. 42(17): e131) and filamentous fungi such as Trichoderma reesei (Liu et al., 2015, Efficient genome editing in filamentous fungus Trichoderma reesei using the CRISPR/Cas9 system. Cell Discovery 1) and Aspergillus niger (Nødvig et al., 2015, A CRISPR-Cas9 System for Genetic Engineering of Filamentous Fungi. PLoS ONE 10(7): e0133085).

The power of the Cas9 system lies in its simplicity to target and edit up to a single base pair in a specific gene of interest. In addition, it is possible to target multiple genes for modification (multiplexing) in a single reaction, generate insertions and deletions, as well as silence or activate genes. In 2012, The CRISPR-Cas9 protein was shown to be a dual-RNA guided endonuclease protein (Jinek et al., 2012, A programmable dual-RNA-guided DNA endonuclease in adaptive bacterial immunity, Science 337(6096): 816-21). Further development for utilization the CRISPR-Cas9 as a genome editing tool has led to the engineering of a single guided RNA molecule that guides the endonuclease to its DNA target. The single guide RNA retains the critical features necessary for both interaction with the Cas9 protein and further targeting to the desired nucleotide sequence. When complexed with the RNA molecule, the Cas9 protein will bind DNA sequence and create a double stranded break using two catalytic domains. When engineered to contain a single amino acid mutation in either catalytic domain, the Cas9 protein functions as a nickase, a variant protein with single stranded cleavage activity. Genome editing in Clostridium cellulyticum via CRISPR-Cas9 nickase was recently demonstrated by Xu et al. (Xu et al., 2015, Efficient Genome Editing in Clostridium cellulolyticum via CRISPR-Cas9 Nickase, Appl. Environ. Microbiol. 81(13): 4423-4431). A basic systematic analysis of essential genes in Bacillus subtilis using a CRISPR-based knockdown approach has been shown (Peters et al., 2016, Cell 165: 1493-1506).

New methods for the successful transient or permanent reduction or complete elimination of the expression of one or more specific gene(s) in industrially employed microorganism host cells are sought. Such host cells are desirable because specific gene-silencing may lead to interesting improved properties, e.g., better transformation efficiency, higher product yield or productivity etc.

### SUMMARY OF THE INVENTION

In a first aspect, the invention provides methods for improving at least one property of a microorganism host cell by repressing the expression of one or more genome target sequence of interest, said methods comprising the steps of:
I) providing a microorganism host cell comprising one or more genome target sequence of interest to be repressed, wherein each target sequence comprises or is flanked by a functional PAM sequence for a Class-II Cas9 enzyme;
II) transforming said host cell with:
   a) a polynucleotide encoding a nuclease-null variant of the Class-II Cas9 enzyme,
   b) a polynuceotide encoding a single-guide RNA or a guide RNA complex for each target sequence to be repressed, said single-guide RNA or guide RNA complex comprising:
      i) a first RNA sequence comprising 20 or more nucleotides that are at least 80% complementary to and capable of hybridizing to the at least one genome target sequence to be repressed and comprising a tracr mate sequence, and
      ii) a second RNA sequence comprising a tracr sequence complementary to and capable of hybridizing with the tracr mate sequence;
         wherein the 20 or more nucleotides of the first RNA sequence hybridize with the one or more genome target sequence and wherein the nuclease-null variant Class-II Cas9 enzyme interacts with the single-guide RNA or the guide RNA complex and binds to the one or more genome target sequence, thereby repressing its expression and improving at least one property of said host cell.

In a second aspect, the invention provides microorganism host cells having at least one improved property, wherein the expression of one or more genome target sequence of interest is repressed, said host cells comprising:
a) one or more repressed genome target sequence of interest, wherein each target sequence comprises or is flanked by a functional PAM sequence for a Class-II Cas9 enzyme;
b) a polynucleotide encoding a nuclease-null variant of the Class-II Cas9 enzyme,
c) a polynuceotide encoding a single-guide RNA or a guide RNA complex for each repressed target sequence, said single-guide RNA or guide RNA complex comprising:
   i) a first RNA sequence comprising 20 or more nucleotides that are at least 80% complementary to and capable of hybridizing to the at least one repressed genome target sequence and comprising a tracr mate sequence, and
   ii) a second RNA sequence comprising a tracr sequence complementary to and capable of hybridizing with the tracr mate sequence;
      wherein the 20 or more nucleotides of the first RNA sequence hybridize with the one or more genome target sequence and wherein the nuclease-null variant Class-II Cas9 enzyme interacts with the single-guide RNA or the guide RNA complex and binds to the one or more genome target sequence, thereby repressing its expression and improving at least one property of said host cell.

### BRIEF DESCRIPTION OF DRAWINGS

FIG. 1 shows a schematic circular plasmid map of pMOL3188 with targets of primers 1-10 indicated.

FIG. 2 shows a schematic linear plasmid map of pMOL3198 with targets of primers 11-22 indicated.

### DEFINITIONS

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

Mature polypeptide: The term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity” or “sequence complementarity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity (or corresponding sequence complementarity) between two nucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the nobrief option) is used as the percent identity and is calculated as follows:

(Identical Deoxyribonucleotides×100)/(Length of Alignment−Total Number of Gaps in Alignment)

To determine the % complementarity of two complementary sequences, one of the two sequences needs to be converted to its complementary sequence before the % complementarity can then be calculated as the % identity between the first sequence and the second converted sequences using the above-mentioned algorithm.

Variant: The term “variant” means a polypeptide comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position, e.g., 1-5 amino acids, adjacent to the amino acid occupying a position.

### DETAILED DESCRIPTION OF THE INVENTION

In a first aspect, the invention provides methods for improving at least one property of a microorganism host cell by repressing the expression of one or more genome target sequence of interest, said methods comprising the steps of:
I) providing a microorganism host cell comprising one or more genome target sequence of interest to be repressed, wherein each target sequence comprises or is flanked by a functional PAM sequence for a Class-II Cas9 enzyme;
II) transforming said host cell with:
   c) a polynucleotide encoding a nuclease-null variant of the Class-II Cas9 enzyme,
   d) a polynuceotide encoding a single-guide RNA or a guide RNA complex for each target sequence to be repressed, said single-guide RNA or guide RNA complex comprising:
      i) a first RNA sequence comprising 20 or more nucleotides that are at least 80% complementary to and capable of hybridizing to the at least one genome target sequence to be repressed and comprising a tracr mate sequence, and
      ii) a second RNA sequence comprising a tracr sequence complementary to and capable of hybridizing with the tracr mate sequence;
         wherein the 20 or more nucleotides of the first RNA sequence hybridize with the one or more genome target sequence and wherein the nuclease-null variant Class-II Cas9 enzyme interacts with the single-guide RNA or the guide RNA complex and binds to the one or more genome target sequence, thereby repressing its expression and improving at least one property of said host cell.

In a second aspect, the invention provides microorganism host cells having at least one improved property, wherein the expression of one or more genome target sequence of interest is repressed, said host cells comprising:
a) one or more repressed genome target sequence of interest, wherein each target sequence comprises or is flanked by a functional PAM sequence for a Class-II Cas9 enzyme;
b) a polynucleotide encoding a nuclease-null variant of the Class-II Cas9 enzyme,
c) a polynuceotide encoding a single-guide RNA or a guide RNA complex for each repressed target sequence, said single-guide RNA or guide RNA complex comprising:
   iii) a first RNA sequence comprising 20 or more nucleotides that are at least 80% complementary to and capable of hybridizing to the at least one repressed genome target sequence and comprising a tracr mate sequence, and
   iv) a second RNA sequence comprising a tracr sequence complementary to and capable of hybridizing with the tracr mate sequence;
      wherein the 20 or more nucleotides of the first RNA sequence hybridize with the one or more genome target sequence and wherein the nuclease-null variant Class-II Cas9 enzyme interacts with the single-guide RNA or the guide RNA complex and binds to the one or more genome target sequence, thereby repressing its expression and improving at least one property of said host cell.

### Prokaryotic Host Cells

The present invention also relates to recombinant prokaryotic host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described herein.

The term “host cell” encompasses any progeny of a parent cell that is not identical to the parent cell due to mutations that occur during replication.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

In a preferred embodiment of the present invention, the Bacillus host cell is chosen from the group of Bacillus species consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294).

The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

### Fungal Host Cells

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phiebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium suiphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Nati. Acad. Sci. USA 75: 1920.

### Genome Target Sequence

The at least one genome target sequence to be modified by the methods of the invention is at least 20 nucleotides in length in order to allow its hybridization to the corresponding 20 nucleotide sequence of the guide RNA. The at least one genome target sequence to be modified can be located anywhere in the genome but will often be within a coding sequence or open reading frame.

The at least one genome target sequence to be modified need to have a suitable protospacer adjacent motif (PAM) located next to it to allow the corresponding nuclease-null variant Class-II Cas9 enzyme to bind a nick the target.

For an overview of other PAM sequences, see, for example, Shah et al, 2013, Protospacer recognition motifs, RNA Biol. 10(5): 891-899.

In a preferred embodiment of the invention, the improved property is an improved transformation efficiency, a reduced protease expression, and/or an improved productivity or yield of a heterologous polypeptide produced by said host cell; preferably the heterologous polypeptide is one or more enzyme selected from the group consisting of a hydrolase, isomerase, ligase, lyase, oxidoreductase, or a transferase; most preferably the enzyme is an alpha-amylase, alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, green fluorescent protein, glucano-transferase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or a xylanase.

Preferably, if the microorganism host cell is a Bacillus host cell, the one or more genome target sequence of interest to be repressed comprises the mecA and/or the yjbH gene or homologues thereof, as exemplified herein. Other preferred genome target sequences of interest to be repressed comprise protease-encoding genes, especially cytosolic, secreted or membrane-bound proteases that, if expressed, may degrade a recombinantly produced polypeptide.

In a preferred embodiment of the invention, the one or more genome target sequence to be repressed comprises at least 20 nucleotides; preferably the one or more genome target sequence to be repressed is comprised in an open reading frame encoding a polypeptide.

Nuclease-Null Variant Class-II Cas9 Several Class-II Cas9 analogues or homologues are known and more are being discovered as the scientific interest has surged over the last few years; a review is provided in Makarova et al., 20015, An updated evolutionary classification of CRISPRCas systems, Nature 13: 722-736.

The Cas9 enzyme of Streptomyces pyogenes is a model Class-II Cas9 enzyme and it is to-date the best characterized. A variant of this enzyme was developed which has only one active nuclease domain (as opposed to the two active domains in the wildtype enzyme) by substituting a single amino acid, aspartic acid for alanine, in position 10: D10A. Another variant of this enzyme was developed which has only one active nuclease domain (as opposed to the two active domains in the wildtype enzyme) by substituting a single amino acid, histidine for alanine, in position 840: H840A. The doubly substituted (D10A, H840A) variant is a nuclease-null variant. It is expected that other Class-II Cas9 enzymes may be modified similarly.

Accordingly, in a preferred embodiment, the nuclease-null variant of the Class-II Cas9 enzyme comprises a substitution in the amino acid position corresponding to position 10 in the Streptomyces pyogenes Cas9 amino acid sequence; preferably the nuclease-null variant of the Class-II Cas9 enzyme comprises a substitution of aspartic acid for alanine, D10A, in the Streptomyces pyogenes Cas9 amino acid sequence. It is also preferred that the nuclease-null variant of the Class-II Cas9 enzyme comprises a substitution in the amino acid position corresponding to position 840 in the Streptomyces pyogenes Cas9 amino acid sequence; preferably the nuclease-null variant of the Class-II Cas9 enzyme comprises a substitution of histidine for alanine, H840A, in the Streptomyces pyogenes Cas9 amino acid sequence.

In a preferred embodiment, the one or more genome target sequence of interest is transiently repressed by a temperature-sensitive nuclease-null variant of the Class-II Cas9 enzyme; preferably the temperature-sensitive nuclease-null variant of the Class-II Cas9 enzyme is unable to bind to the one or more genome target sequence at a temperature above 35° C.; preferably above 36° C.; above 37° C.; above 38° C.; above 39° C.; above 40° C.; above 41° C.; above 42° C.; above 43° C.; above 44° C.; or most preferably above 45° C.

### Guide RNA

The guide RNA in CRISPR-Cas9 genome editing constitutes the re-programmable part that makes the system so versatile. In the natural S. pyogenes system the guide RNA is actually a complex of two RNA polynucleotides, a first crRNA containing about 20 nucleotides that determine the specificity of the Cas9 enzyme as well as the tracr RNA which hybridizes to the crRNA to form an RNA complex that interacts with Cas9. See Jinek et al., 2012, A programmable dual-RNA-guided DNA endonuclease in adaptive bacterial immunity, Science 337: 816-821. The terms crRNA and tracrRNA are used interchangeably with the terms tracr-mate RNA and tracr RNA herein.

Since the discovery of the CRISPR-Cas9 system single polynucleotide guide RNAs have been developed and successfully applied just as effectively as the natural two part guide RNA complex.

In a preferred embodiment, the single-guide RNA or RNA complex comprises a first RNA comprising 20 or more nucleotides that are at least 85% complementary to and capable of hybridizing to the one or more genome target sequence; preferably the 20 or more nucleotides are at least 90%, 95%, 97%, 98%, 99% or even 100% complementary to and capable of hybridizing to the one or more genome target sequence.

In another preferred embodiment, the Bacillus host cell comprises a single-guide RNA comprising the first and second RNAs in the form of a single polynucleotide and wherein the tracr mate sequence and the tracr sequence form a stem-loop structure when hybridized with each other.

### Multiplexing

In a preferred embodiment, wo or more genome target sequences in the host cell are repressed.

### Polynucleotides

The techniques used to isolate or clone a polynucleotide are known in the art and include isolation from genomic DNA or cDNA, or a combination thereof. The cloning of the polynucleotides from genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as a mature polypeptide coding sequence, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising certain polynucleotides operably linked to one or more control sequences that direct the expression of the coding sequence.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis clyllIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCI B 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMR1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sam brook et al., 1989, supra).

### Methods of Production

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### EXAMPLES

### Materials and Methods for Examples 1-5:

### Media

Bacillus strains were grown on LB agar plates (10 g/l Tryptone, 5 g/l yeast extract, 5 g/l NaCl, 15 g/l agar) or in LB liquid medium (10 g/l Tryptone, 5 g/l yeast extract, 5 g/l NaCl). In some experiments the LB was supplemented with phosphate, glucose and starch (LBPGS) by adding 5 g/l starch, 20 ml/l of 20% glucose and 10 ml/l of 1 M K₂PO₄.

To select for erythromycin resistance, agar media were supplemented with 1 μg/ml erythromycin and liquid media were supplemented with 2 μg/ml erythromycin. To select for chloramphenicol resistance, agar and liquid media were supplemented with 6 μg/ml chloramphenicol.

To screen for protease phenotypes agar plates were supplemented with 1% skim milk to allow halos to form around the colonies that produces protease.

Spizizen 1-xyl medium consists of 1× Spizizen salts (6 g/l KH₂PO₄, 14 g/l K₂HPO₄, 2 g/l (NH₄)₂SO₄, 1 g/l sodium citrate, 0.2 g/l MgSO₄, pH 7.0), 1% xylose, 0.1% yeast extract, and 0.02% casein hydrolysate.

Spizizen II-xyl medium consists of Spizizen 1-xyl medium supplemented with 0.5 mM CaCl₂, and 2.5 mM MgCl₂ and 2 mM EGTA.

### Strains

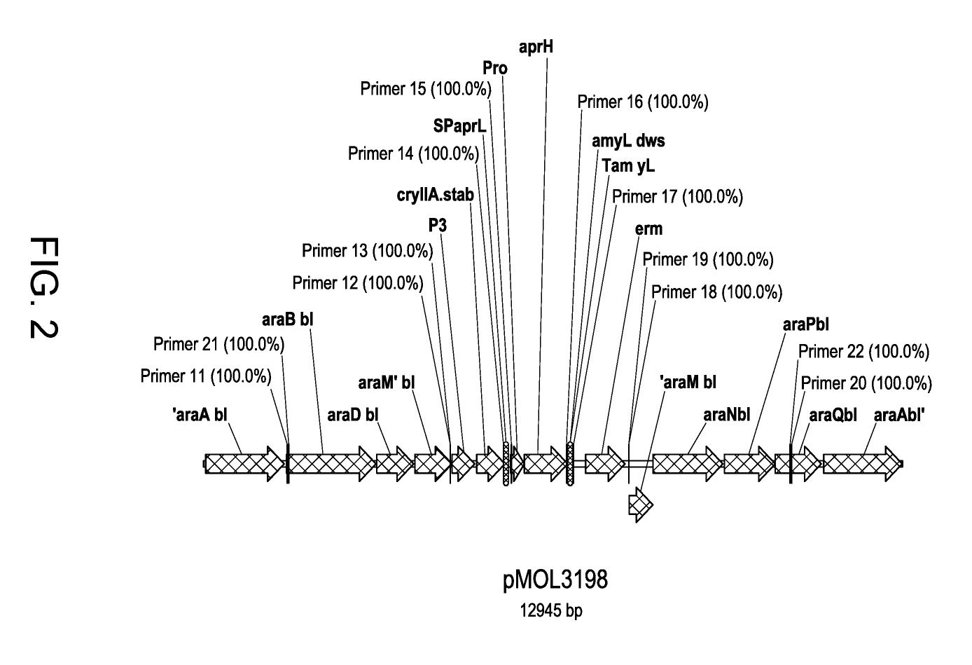

JA1622: This strain is the B. subtilis 168 derivative JA578 described in WO 02/00907 with a disrupted spollAC gene (sigF). The genotype is: amyE::repF (pE194), spollAC.
MOL3188: JA1622+pMOL3188
AEB708: B. subtilis strain where the M.Bli1904II DNA methyltransferase gene from B. licheniformis is inserted at the amyE locus by chromosomal transformation WO 2015/004013
MOL3190: AEB1517 with pMOL3188
SJ8039: B. subtilis conjugation donor PP289-5+pSJ8017 described in WO 2007/138049. The plasmid is for deletion of catL in B. licheniformis
SJ1904: This strain is a B. licheniformis strain described in WO 2008/066931. The gene encoding the alkaline protease (aprL) is inactivated.
MOL3026: SJ1904 with a catL deletion (described in example 2)
TaHy9: B. licheniformis SJ1904 with a mecA deletion as described WO 2015/004013
MOL3027: The TaHy9 strain with a catL deletion (described in example 2)
MOL3053: B. licheniformis strain described in WO 2015/004013. The protease gene aprH integrated at the arabinose operon.
MOL3192: MOL3026+forD-cas9d (described in example 3)
MOL3193: MOL3027+forD-cas9d (described in example 3)
MOL3198: MOL3027+ara:aprH-erm (described in example 4 and FIG. 2).

### Plasmids

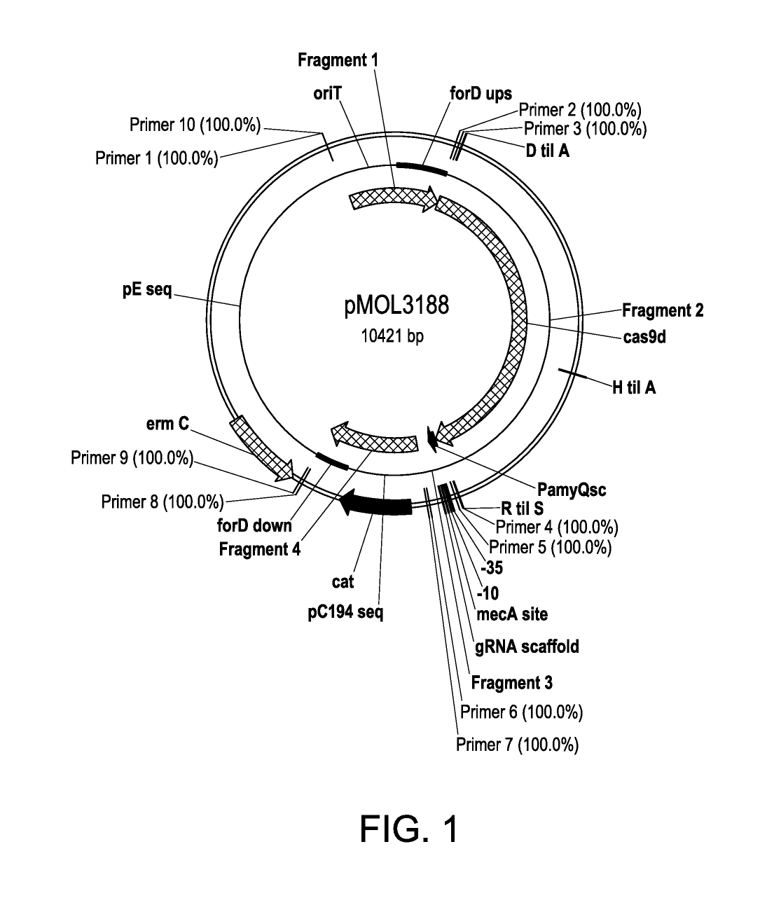

pC194: Plasmid isolated from Staphylococcus aureus (Horinouchi and Weisblum, 1982).
pE194: Plasmid isolated from Staphylococcus aureus (Horinouchi and Weisblum, 1982).
pUB110: Plasmid isolated (McKenzie et al., 1987)
pMOL3188: Described in Experiment 1 and FIG. 1
pSJ8017: Described in WO 2007/138049.

### Molecular Biological Methods

DNA manipulations and transformations were performed by standard molecular biology methods as described in:
Sambrook et al. (1989): Molecular cloning: A laboratory manual. Cold Spring Harbor laboratory, Cold Spring Harbor, N.Y.
Ausubel et al. (eds) (1995): Current protocols in Molecular Biology. John Wiley and Sons.
Harwood and Cutting (eds) (1990): Molecular Biological Methods for Bacillus. John Wiley and Sons.

Enzymes for DNA manipulation were obtained from New England Biolabs, Inc. and used essentially as recommended by the supplier.

Competent cells and transformation of B. subtilis was obtained as described in Yasbin et al., 1975, Transformation and transfection in lysogenic strains of Bacillus subtilis: evidence for selective induction of prophage in competent cells, J. Bacteriol. 121: 296-304.

Genomic DNA was prepared from several erythromycin sensitive isolates above accordingly to the method previously described (Pitcher et al., supra) or by using the commercial available QIAamp DNA Blood Kit from Qiagen.

TABLE 1
Sequence overview for examples 1-5.

| | SEQ | |
|---|---|---|
| | ID | Nucleotide sequences |
| | NO | 5′ → 3′ |
| Fragment 1 | 1 | See sequence list |
| Fragment 2 | 2 | See sequence list |
| Fragment 3 | 3 | See sequence list |
| Fragment 4 | 4 | See sequence list |
| Fragment 5 | 5 | See sequence list |
| Fragment 6 | 6 | See sequence list |
| Primer 1 | 7 | ccgactgcgcaaaagacataatcga |
| | | ctctagaggatccccgggtaccgag |
| Primer 2 | 8 | cgatgctgtattttttgtccattcg |
| | | cttccctcctcatttttagcgcgtc |
| Primer 3 | 9 | atggacaaaaaatacagcatcggc |
| Primer 4 | 10 | cacagcggacagtctggacagcatc |
| | | aatcgcctcccagttgtgacag |
| Primer 5 | 11 | tgctgtccagactgtccgctgtg |
| Primer 6 | 12 | cccactttatccaatattcgttcct |
| | | taatttcatgaacaatcttcattc |
| Primer 7 | 13 | ggaacgaatattggataaagtggg |
| Primer 8 | 14 | gtgtgcctattttttgtgaatcgac |
| | | ctgcaggcatgcaagcttgtttatg |
| Primer 9 | 15 | tcgattcacaaaaaataggcacacg |
| Primer 10 | 16 | gtcgattatgtcttttgcgcagtcg |
| Primer 11 | 17 | gtgcagttctcgtcgatgtccgg |
| Primer 12 | 18 | ggtcaagacattgatcaagcgcttc |
| Primer 13 | 19 | gaagcgcttgatcaatgtcttgacc |
| | | gcaacgttcgcagatgctgctgaag |
| Primer 14 | 20 | ccataatacataattttcaaactg |
| Primer 15 | 21 | gaaaaatatttaattggctttaatg |
| | | agcagg |
| Primer 16 | 22 | cgcgttgccgcttctgcattgacaa |
| | | g |
| Primer 17 | 23 | gttcaataatcgcatccgattgcag |
| Primer 18 | 24 | ctgcgcaaaagacataatcgattc |
| Primer 19 | 25 | gaatcgattatgtcttttgcgcaga |
| | | tgctggttctcgaccattcaagg |
| Primer 20 | 26 | cgtagacggcaagcccgtatcc |
| Primer 21 | 27 | ggtgaggaaatcgcgacagctg |
| Primer 22 | 28 | ccattgaagaaaacaagagcgtc |
| pMOL3188 | 29 | See sequence list |
| Cas9d | 30 | See sequence list |

### Example 1. Cas9d and Guide-RNA Vector Targeting mecA in B. licheniformis

It has been described earlier that a mecA deletion in Bacillus licheniformis will result in more efficient competence development of the strain (WO 2015/004013). However, a deletion of mecA is not always desirable, as the mecA-deficient strain shows slightly altered growth characteristics and decreased potential for enzyme expression. In Examples 1-5 an alternative repression of the mecA gene is shown, where a CRISPR-inhibition system is exploited.

By performing SOE-PCR with the primers, templates and synthetic DNA (LifeTechnologies) listed in below, a plasmid was assembled holding a synthetic version of the cas9d gene (SEQ ID NO: 30), encoding the Cas9d polypeptide which has two amino acids substituted: D10A and H840A compared to the wildtype Cas9, thereby inactivating both nuclease activities. The cas9d gene is controlled by the forD promoter from B. licheniformis. The consensus version of the promoter from amyQ (Bacillus amyloliquefaciens) controls the expression of the guideRNA (gRNA) with the recognition site for the mecA promoter: tcttacaaagaagggaaggt (nucleotides 95 to 115 in fragment 3; SEQ ID NO: 3), followed by the Cas9 PAM sequence: gtt. The cat marker for selection of chromosomal insertion was amplified from the plasmid pC194 and the plasmid backbone holding the origin and replication functions was amplified from pE194. The oriT region from plasmid pUB110 was inserted to allow for conjugation as described earlier (WO 96/29418). The complete pMOL3188 sequence is listed in SEQ ID NO: 29. The plasmid map of pMOL3188 is shown in FIG. 1.

TABLE 2
SOE-PCR strategy for assembling the plasmid pMOL3188
1. PCRs

| | | Fragment/ | Size |
|---|---|---|---|
| Annotation | Donor | primers | (bp) |
| oriT | pUB110 | Synthetic | 1144 |
| PforD | B. licheniformis SJ1904 | fragment 1 | |
| cas9d gene | Streptococcus pyogenes | Synthetic | 4107 |
| codon optimized | | fragment 2 | |
| PamyQsc | B. amyliquefaciens | Synthetic | 263 |
| gRNA- mecA | Streptococcus pyogenes | fragment 3 | |
| cat marker | pC194 | Synthetic | 1176 |
| forD downstream | B. licheniformis SJ1904 | fragment 4 | |
| Plasmid origin and | pE194 | Primer 1 & 2 | 3731 |
| erm | | | |

### Example 2. Deletion of the catL Gene in Strains SJ1904 and TaHy9

The plasmid pSJ8017 was transformed into the two B. licheniformis strains SJ1904 and TaHy9 by conjugation from the B. subtilis donor strain SJ8039. The conjugation was performed as described earlier ONO 2007/138049). After conjugation, the plasmid was integrated and excised at the catL locus on the chromosome of SJ1904 and TaHy9 to replace the functional catL gene with the deleted version delivered by the plasmid pSJ8017. The catL deleted B. licheniformis strains SJ1904 and TaHy9 were preserved as MOL3026 and MOL3027, respectively.

### Example 3. Conjugation of pMOL3188 into forD in Strains MOL3026 and MOL3027

The SOE-PCR assembled plasmid pMOL3188 was transformed into competent B. subtilis JA1622, selecting for resistance against erythromycin and chloramphenicol. Transformants were tested by restriction digest analysis and DNA sequencing. The strain holding plasmid pMOL3188 was named MOL3188. This strain was used as a donor for plasmid transfer by conjugation.

The temperature-sensitive plasmid pMOL3188 was integrated into the genomes of B. licheniformis MOL3026 and MOL3027 by chromosomal integration and excision according to the method previously described (U.S. Pat. No. 5,843,720). B. licheniformis transformants containing plasmid pMOL3188 were grown on LBPGS selective medium (cam) at 50° C. Desired integrants were chosen based on their ability to grow on LBPGS chloramphenicol selective medium at 50° C. Integrants were then grown without selection in LBPGS medium at 34° C. to allow excision of the integrated plasmid. Cells were plated on LBPGS plates and screened for erythromycin-sensitivity and chloramphenicol resistance.

Genomic DNA was prepared from several erythromycin sensitive isolates. Genomic PCR confirmed insertion of the cas9d-gRNA(mecA) cassette in the forD locus of MOL3026 and MOL3027. The resulting verified strains was designated MOL3192 and MOL3193, respectively.

### Example 4: Construction of an aprH Expression Cassette with an erm Marker in the Arabinose Operon of Strain MOL3027

An expression cassette with the alkaline protease from Bacillus clausii aprH was inserted together with the erm marker into the ara locus of the chromosome of MOL3027 described above. The SOE product was assembled as listed in the table below and transformed directly into the competent MOL3027 as described above. Colonies on erm plates were verified with PCR and frozen as MOL3198.

TABLE 3
SOE-PCR strategy for inserting the aprH gene and erm gene
into the ara operon of B. licheniformis MOL3027

| 1. PCRs | | | | SOE-PCR | |
|---|---|---|---|---|---|
| | | Fragment/ | Size | | Size |
| Annotation | Donor | primers | (bp) | Primers | (bp) |
| ara fragment | SJ1904 | 11 & 12 | 3061 | 21 & 22 | 9382 |
| upstream | chr DNA | | | | |
| P3 promoter | MOL3053 | 13 & 14 | 1003 | | |
| Signal peptide | SJ1904 | Fragment 5 | 180 | | |
| from alkaline | chr DNA | synthetic | | | |
| protease aprL | | | | | |
| aprH | MOL3053 | 15 & 16 | 1051 | | |
| amyL terminator | SJ1904 | Fragment 6 | 160 | | |
| | chr DNA | synthetic | | | |
| Erm | pE194 | 17 & 18 | 1043 | | |
| ara fragment | SJ1904 | 19 & 20 | 3047 | | |
| downstream | chr DNA | | | | |

### Example 5: Transformation Efficiency of Strains MOL3192 and MOL3193 with Constitutive CRISPRi Repression of mecA

The B. licheniformis strains MOL3026, MOL3027, MOL3192 and MOL3193 were spread onto LB agar plates to obtain confluent growth after incubation at 37° C. overnight.

After overnight incubation, approximately 2-3 ml of Spizizen I-xyl medium was added to each plate. Cells were scraped using sterile spreaders and transferred into 15 ml Falcon 2059 tubes. Approximately 500 μl of each culture-suspension was used to inoculate 50 ml Spizizen I-xyl medium. Growth was monitored using a Klett densitometer. At each cell density corresponding to Klett unit 140, 160, 180, and 200, 250 μl of the culture plus 250 μl Spizizen II-xyl medium containing 2 mM EGTA was added to a Falcon 2059 tube.

One microgram of transforming B. licheniformis MOL3147 chromosomal DNA containing an erm resistance expression cassette integrated at the ara locus; was added to each tube. Tubes were incubated at 37° C. on a rotational shaker set at 250 rpm for 1 hour. Transformation reactions were plated to LB agar plates containing 1 μg/ml of erm. Colonies were counted the following day to determine transformation efficiency.

As can been seen in the table below, the MOL3192 strain which comprises the CRISPRi cassette with the guide-RNA directed against mecA can be transformed with genomic DNA, unlike the control strain MOL3026. The two positive control strains MOL3027 and MOL3193 both have the mecA deletion and can be transformed with gDNA as expected and as described earlier for mecA-deleted strains in WO2015/004013.

TABLE 4
Transformation frequency as cfu/μg of genomic DNA (gDNA).

| | | | MOL3026- | | MOL3027- | |
|---|---|---|---|---|---|---|
| | Control strains | | CRISPRi(mecA) | | CRISPRi(mecA) | |
| | MOL3026 | MOL3027 | MOL3192 | MOL3192 | MOL3193 | MOL3193 |
| DNA | 0 | 750 | 145 | 150 | 580 | 600 |
| gDNA | | | | | | |
| MOL3198 | | | | | | |
| No DNA | 0 | 0 | 0 | 0 | 0 | 0 |

### Materials and Methods for Examples 6-13:

It has previously been shown that inactivation of yjbH in a Bacillus subtilis host cell improved the productivity or yield of an alpha amylase (WO 00/63346). In the following examples we employed CRISPR-interference to silence yjbH expression in a recombinant Bacillus subtilis host cell, thereby improving the productivity or yield of a heterologous alpha amylase.

Examples 6 and 7 below outline the construction of plasmids in this work. Examples 8-12 outline the construction of strains and Example 13 shows the results.

### Strains

Escherichia coli Stellar™ Competent cells (Clontech laboratories, Mountain View, Calif.) were used for routine plasmid constructions and propagation.

Bacillus subtilis RB128 was used as a host for establishing Cas9-based gene silencing; this strain is a Bacillus subtilis A164Δ5 strain (Bacillus subtilis ATCC 6051A deleted at the spollAC, aprE, nprE, amyE, and srfC genes) obtained according to the methods of U.S. Pat. No. 5,891,701. Bacillus subtilis strain RB128 contains a heterologous gene encoding a Bacillus maltogenic amylase.

### Media:

Bacillus strains were grown on TBAB (Tryptose Blood Agar Base, Difco Laboratories, Sparks, Md., USA) or LB agar (10 g/l Tryptone, 5 g/l yeast extract, 5 g/l NaCl, 15 g/l agar) plates or in LB liquid medium (10 g/l Tryptone, 5 g/l yeast extract, 5 g/l NaCl).

To select for erythromycin resistance, agar media were supplemented with 1 μg/ml erythromycin+25 μg/ml lincomycin and liquid media were supplemented with 5 μg/ml erythromycin. To select for chloramphenicol resistance, Both agar and liquid medium were supplemented with 5 μg/ml chloramphenicol.

Spizizen I medium consists of 1× Spizizen salts (6 g/l KH₂PO₄, 14 g/l K₂HPO₄, 2 g/l (NH₄)₂SO₄, 1 g/l sodium citrate, 0.2 g/l MgSO₄, pH 7.0), 0.5% glucose, 0.1% yeast extract, and 0.02% casein hydrolysate.

Spizizen II medium consists of Spizizen I medium supplemented with 0.5 mM CaCl₂, and 2.5 mM MgCl₂.

MRS medium was prepared using 55 g/l Lactobacilli MRS Broth (Becton, Dickinson and Company, Franklin Lakes, N.J.) according to the manufacturer's recommendation.

### Preparation and Transformation of Competent Cells:

B. subtilis RB128 was spread onto LB agar plates to obtain single colonies after incubation at 37° C. overnight. After overnight incubation, one colony was used to inoculate 10 ml of LB medium. The following day, approximately 500 μl of this culture was used to inoculate 50 ml Spizizen I medium. Growth was monitored using a Klett densitometer. Cells were harvested immediately as they entered stationary phase and used to inoculate Spizizen II medium. The Spizizen II culture was grown for an additional 90 minutes. Cells were harvested and either immediately used for transformation or frozen in 500 μl aliquots in 15% glycerol.

To 500 μl of competent cells, 500 μl Spizizen II medium containing 2 mM EGTA was added. Two hundred fifty microliters of cell mixture was then transferred to a Falcon 2059 tube. One microgram of transforming DNA was added to each tube, followed by 250 μl of LB. Two microliters of 50 μg/ml appropriate antibiotic was included in the transformation mix. Tubes were incubated at 34° C. or 37° C. on a rotational shaker set at 250 rpm for 1 hour. Transformation reactions were plated to LB agar plates containing the appropriate antibiotic. Colonies were harvested after 24 hours at 37° C. or after 48 hours at 34° C.

### Example 6: Construction of Plasmid pBM376a

A synthetic DNA fragment containing the scBAN promoter minus its ribosome binding site, plus the yjbH guide RNA targeting the DNA top strand was obtained from GeneArt (Thermo Fischer Scientific, Grand Island, N.Y.); the DNA sequence is shown below:

SEQ ID NO: 31:
Aagctttgctgtccagactgtccgctgtgtaaaaaaaaggaataaagggg
ggttgacattattttactgatatgtataatataatttgtataagaaaatg
tacaaacatctgggaaatacgttttagagctagaaatagcaagttaaaat
aaggctagtccgttatcaacttgaaaaagtggcaccgagtcggtgcttta
agctt

This fragment was cloned into temperature-sensitive Bacillus/E. coli shuttle vector pBM354 as follows: The primers below were used for amplification of the synthetic DNA.

Primer 1216467 (SEQ ID NO: 32):
5′ cctcgaggtcgacggtatcgataagctttgctgtccagactgtc
Primer 1216468 (SEQ ID NO: 33):
5′ gctgcaggaattcgatatcaagcttaaagcaccgactcggtgcc

The DNA fragment was amplified using Phusion Hot Start II polymerase (Thermo Scientific, Grand Island, N.Y.). The PCR amplification reaction mixture contained 1 μl of 25 ng/ul synthetic DNA, 0.5 μl of sense primer (50 μmol/μl), 0.5 μl of anti-sense primer (50 μmol/μl), 5 μl of 10× Phusion HF PCR buffer, 1 μl of dNTP mix (10 mM each), 36.5 μl water, and 0.5 μl (2.0 U/μl) DNA polymerase mix. An Eppendorf Mastercycler thermocycler was used to amplify the fragment with the following settings: One cycle at 98° C. for 30 seconds; 25 cycles each at 98° C. for 10 seconds, 58° C. for 20 seconds, 72° C. for 20 seconds; one cycle at 72° C. for 5 minutes; and 4° C. hold.

The 205 bp PCR product was purified from a 1.8% agarose (Amresco, Solon, Ohio) gel with 1×TBE buffer using the Qiagen QIAquick Gel Extraction Kit (Qiagen, Inc., Valencia, Calif.) according to the manufacturer's instructions.

The 205 bp PCR fragment comprising the scBAN promoter minus the ribosome binding site, plus the yjbH guide RNA was cloned into plasmid pBM354, which had previously been digested with restriction enzyme HindIII, using Clontech In-Fusion HD Cloning System (Clontech laboratories, Inc., Mountain View, Calif.) according to the manufacturer's instructions.

A 2 μl aliquot of the In-fusion mix was used to transform E. coli Stellar™ cells according to the manufacturer's instructions. Plasmid DNA was prepared from E. coli transformants. DNA sequencing of one such transformant was identified as having correct DNA sequence and designated pBM376a.

### Example 7: Construction of Plasmid pBM375C

A synthetic DNA fragment containing the scBAN promoter minus its ribosome binding site, plus the yjbH guide RNA targeting the DNA bottom strand was obtained from GeneArt (Thermo Fischer Scientific, Grand Island, N.Y.); the DNA sequence is shown below as SEQ ID NO: 34:

aagctttgctgtccagactgtccgctgtgtaaaaaaaaggaataaagggg
ggttgacattattttactgatatgtataatataatttgtataagaaaatg
accctttatgtcctgaatgcgttttagagctagaaatagcaagttaaaat
aaggctagtccgttatcaacttgaaaaagtggcaccgagtcggtgcttta
agctt

The fragment was cloned into temperature-sensitive Bacillus/E. coli shuttle vector pBM354 as follows:

The primers below were used for amplification of the synthetic DNA:

Primer 1216467 (SEQ ID NO: 32):
5′- cctcgaggtcgacggtatcgataagctttgctgtccagactgtc
Primer 1216468 (SEQ ID NO: 33):
5′- gctgcaggaattcgatatcaagcttaaagcaccgactcggtgcc

The DNA fragment was amplified using Phusion Hot Start II polymerase (Thermo Scientific, Grand Island, N.Y.). The PCR amplification reaction mixture contained 1 μl of 25 ng/ul synthetic DNA, 0.5 μl of sense primer (50 μmol/μl), 0.5 μl of anti-sense primer (50 μmol/μl), 5 μl of 10× Phusion HF PCR buffer, 1 μl of dNTP mix (10 mM each), 36.5 μl water, and 0.5 μl (2.0 U/μl) DNA polymerase mix. An Eppendorf Mastercycler thermocycler was used to amplify the fragment with the following settings: One cycle at 98° C. for 30 seconds; 25 cycles each at 98° C. for 10 seconds, 58° C. for 20 seconds, 72° C. for 20 seconds; one cycle at 72° C. for 5 minutes; and 4° C. hold.

The 205 bp PCR product was purified from a 1.8% agarose (Amresco, Solon, Ohio) gel with 1×TBE buffer using the Qiagen QIAquick Gel Extraction Kit (Qiagen, Inc., Valencia, Calif.) according to the manufacturer's instructions.

The 205 bp PCR fragment comprising the scBAN promoter minus the ribosome binding site, plus the yjbH guide RNA was cloned into plasmid pBM354, which had previously been digested with restriction enzyme HindIII, using Clontech In-Fusion HD Cloning System (Clontech laboratories, Inc., Mountain View, Calif.) according to the manufacturer's instructions.

A 2 μl aliquot of the In-fusion mix was used to transform E. coli Stellar™ cells according to the manufacturer's instructions. Plasmid DNA was prepared from E. coli transformants. DNA sequencing of one such transformant was identified as having correct DNA sequence and designated pBM375c.

### Example 8: Construction of Strain BaC0266

To evaluate use of the type II CRISPR-Cas9 system from Streptococcus pyogenes in Bacillus, we chose to work with a Bacillus strain defective in non-homologous end joining (NHEJ). DNA damage due to double stranded breaks can be repaired in Bacillus via two pathways: error-free homologous recombination (HR) or non-homologous end joining. A double stranded break, induced by CRISPR Cas9 in a strain defective in NHEJ, would be lethal, unless repaired by homologous recombination. Two genes involved in non-homologous end joining (NHEJ) in Bacillus subtilis are annotated as ligD and ku (de Vega, 2013, The minimal Bacillus subtilis nonhomologous end joining repair machinery, PLoS One 8(5): e64232). The ligD gene codes for a multi-functional DNA ligase D, whereas the ku gene codes for a DNA binding protein. A disruption in both genes results in a strain incapable of repairing a double stranded break by means of NHEJ. Since these two genes lie in the same operon in B. subtilis 168 both open reading frames were disrupted simultaneously.

The temperature-sensitive plasmid pBM353 was incorporated into the genome of B. subtilis 168.DELTA.4 by chromosomal integration and excision according to the method previously described (U.S. Pat. No. 5,843,720). B. subtilis 168.DELTA.4 transformants containing plasmid pBM353 were grown on TBAB supplemented with erythromycin/lincomycin at 50° C. to force integration of the vector. Desired integrants were chosen based on their ability to grow on TBAB erythromycin/lincomycin selective medium at 50° C. Integrants were then grown without selection in LB medium at 37° C. to allow excision of the integrated plasmid. Cells were plated on LB plates and screened for erythromycin-sensitivity.

Genomic DNA was prepared from several erythromycin/lincomycin sensitive isolates above accordingly to the method previously described (Pitcher et al., 1989, Rapid extraction of bacterial genomic DNA with guanidium thiocyanate, Letters in Applied Microbiology 8(4): 151-156). Genomic PCR confirmed the disruption of the ykoV (ligD) and ykoU (ku) genes and the resulting strain was designated BaC0266.

### Example 9: Construction of Strain BaC0301

The type II CRISPR-Cas9 system from Streptococcus pyogenes consists of a dual-RNA-guided DNA endonuclease able to target specific genomic sequences. When complexed with a guide RNA molecule, the Cas9 protein will bind DNA sequence and create a double stranded break by means of two catalytic domains. With the introduction of two mutations (D10A and H840A) in the Cas9 catalytic domains, the protein retains its binding capability but loses its catalytic function (Cas9d), when complexed with a specific guide RNA. When bound to DNA the Cas9d-gRNA complex can repress transcription of the target gene.

B. subtilis strain BaC0301 was created for expression of the catalytically-inactive dCas9 as follows:

A linear integration vector-system was used for the expression cloning of the S. pyogenes Cas9d gene. The linear integration construct was a PCR fusion product made by fusion of the cas9 gene between two B. subtilis homologous chromosomal regions along with a strong promoter and a chloramphenicol resistance marker. The fusion was made by SOE PCR. The gene was expressed under the control of a triple promoter system (as described in WO 99/43835), consisting of the promoters from B. licheniformis alpha-amylase gene (amyL), B. amyloliquefaciens alpha-amylase gene (amyQ), and the B. thuringiensis cryIIIA promoter including stabilizing sequence. The gene coding for chloramphenicol acetyl-transferase was used as marker (Diderichsen et al., 1993, A useful cloning vector for Bacillus subtilis, Plasmid 30(3): 312-315). The final gene construct was integrated in the B. subtilis chromosome by homologous recombination into the pectate lyase pel gene locus.

The first fragment designed to amplify the 5′ flanking sequence with homology to the B. subtilis A164 genome plus the DNA sequence for the triple promoter was amplified from B. subtilis A164 strain, BaC0291, in a PCR reaction with the following primers:

1209582 (SEQ ID NO: 35):
5′-ctgcgtgtgcctacagat
1216378 (SEQ ID NO: 36):
5′-gcctattgagtatttcttatccattcggttccctcctcatttttata
gagc

The second fragment designed to contain the S. pyogenes cas9 D10A portion of the gene was PCR amplified from B. subtilis BaC0298 using the following primer pair:

1216377 (SEQ ID NO: 37):
5′-gctctataaaaatgaggagggaaccgaatggataagaaatactcaat
aggc
1217984 (seq ID NO: 38)
5′-catcataatcacttaaacgattaatatctaattcttggtccacatac
atgtc

The third fragment designed to contain the S. pyogenes cas9 H840A portion of the gene was PCR amplified from a synthetic DNA fragment (SEQ ID NO: 39) using the following primer pair:

1213933 (SEQ ID NO: 40):
5′-tgtggaccaagaattagata
1214793 (seq ID NO: 41)
5′-tttcttaatcaaagcagttccaacg

The fourth fragment was designed to amplify the chloramphenicol resistance gene along with the 3′ flanking sequence with homology to the B. subtilis A164 genome was amplified from BaC0291 in a PCR reaction with the following primers:

1213934 (SEQ ID NO: 42):
5′-cgttggaactgctttgattaagaaa
1209587 (SEQ ID NO: 43)
5′-gtgtcgatcagcttcttcagc

The respective DNA fragments were amplified by PCR using Phusion Hot Start II polymerase (Thermo Scientific, Grand Island, N.Y.). The PCR amplification reaction mixture contained approximately 1 μg DNA, 0.5 μl of sense primer (50 μmol/μl), 0.5 μl of anti-sense primer (50 μmol/μl), 5 μl of 10× Phusion HF PCR buffer, 1 μl of dNTP mix (10 mM each), 36.5 μl water, and 0.5 μl (2.0 U/μl) DNA polymerase mix. An Eppendorf Mastercycler thermocycler was used to amplify the fragment with the following settings: One cycle at 98° C. for 30 seconds; 25 cycles each at 98° C. for 10 seconds, 58° C. for 20 seconds, and 72° C. extension time as suggested by the manufacturer; one cycle at 72° C. for 5 minutes; and 4° C. hold. The PCR products were purified from a 1% TBE agarose gel using the Qiagen QIAquick PCR purification Kit (Qiagen, Inc., Valencia, Calif.) according to the manufacturer's instructions.

All four fragments were combined in a single PCR reaction to obtain the final fragment for transformation. The PCR amplification reaction mixture contained approximately 25 ng gel purified DNA, 0.5 μl of sense primer (50 μmol/μl), 0.5 μl of anti-sense primer (50 μmol/μl), 5 μl of 10× Phusion HF PCR buffer, 1 μl of dNTP mix (10 mM each), 36.5 μl water, and 0.5 μl (2.0 U/μl) Phusion Hot Start II polymerase (Thermo Scientific, Grand Island, N.Y.). An Eppendorf Mastercycler thermocycler was used to amplify the fragment with the following settings: One cycle at 98° C. for 30 seconds; 25 cycles each at 98° C. for 10 seconds, 58° C. for 20 seconds, and 72° C. extension time as suggested by the manufacturer; one cycle at 72° C. for 5 minutes, 30 seconds; and 4° C. hold. The PCR products were purified using the QIAquick PCR purification Kit (Qiagen, Inc., Valencia, Calif.) according to the manufacturer's instructions.

One microgram of the purified PCR product was used to transform B. subtilis BaC0266 and transformants were selected on LB-plates containing chloramphenicol (6 μg/ml). One transformant identified by genomic PCR and further DNA sequencing of the S. pyogenes cas9 D10A H840A gene was chosen and named BaC0301.

### Example 10: Construction of Strain BaC0303

One microgram of BaC0301 genomic DNA was used to transform B. subtilis RB128 competent cells and transformants were selected on LB-plates containing chloramphenicol (6 μg/ml). One transformant was isolated and validated by means of SDS-PAGE protein analysis as follows. To examine expression of the Cas9d (D10A, H840A) protein, BaC0303 culture was grown overnight in LB medium at 37° C. The following day, one ml from the overnight culture was harvested, and cells were lysed in Urea Sample buffer using lysing Matrix B (MP Biomedicals, Santa Ana, Calif.). Ten milliliters of Urea Sample buffer consists of 1 ml 10% SDS, 5.4 g urea, 250 μl 1 M Tris-HCl, 20 μl 0.5 M EDTA, pH 8.0, 500 μI beta-mercaptoethanol. Cell-free lysates were subjected to SDS-PAGE using 4-15% TGX Criterion protein gels (Bio-Rad Laboratories, Hercules. Calif.). Expression of the Cas9d (D10A, H840A) variant was validated by comparison to the untransformed RB128 lysate.

### Example 11. Construction of Strain BaC0306

To create a strain capable of silencing the B. subtilis BaC0303 yjbH gene we first prepared naturally competent cells and froze them in five hundred microliter aliquots at −80° C. in 15% glycerol. Then, 500 μl of Spizizen II medium containing 2 mM EGTA was added to a frozen aliquot, after which 250 μl of thawed competent cells was moved to a Falcon tube. One microgram of plasmid pBM375c, 250 μl LB and 2 μl 50 mg/ml erythromycin were added to the Falcon tube. Cells were grown on a rotational shaker set at 250 rpm 34° C. for 2 hours. After 2 hours, the transformation mixture was plated to agar plates containing 25 μg/ml of erythromycin and 1 μg/ml of lincomycin. Plates were put at 34° C. for 2 days. After two days, one individual colony was chosen and named B. subtilis BaC0306.

### Example 12. Construction of Strain BaC0307

To create a strain capable of silencing the B. subtilis BaC0303 yjbH gene we prepared naturally competent cells. Five hundred microliter aliquots of the competent cells were frozen at −80° C. in 15% glycerol. Prior to transformation, 500 μl of Spizizen II medium containing 2 mM EGTA was added to a frozen aliquot, after which 250 μl was moved to a Falcon tube. One microgram of plasmid pBM376, 250 μl LB and 2 μl 50 mg/ml erythromycin were added to the Falcon tube. Cells were grown on a rotational shaker set at 250 rpm 34° C. for 2 hours. After 2 hours, the transformation mixture was plated to agar plates containing 25 μg/ml of erythromycin and 1 μg/ml of lincomycin. Plates were put at 34° C. for 2 days. After two days, one individual colony was chosen and named B. subtilis BaC0307.

### Example 13. CRISPR Interference (CRISPRi) in Bacillus subtilis

To demonstrate the CRISPRi system in Bacillus subtilis, we targeted the transcriptional repression of the yjbH gene in the maltogenic amylase-producing B. subtilis strain RB128. We expected that successful “silencing” of the yjbH gene would result in increased Novamyl activity.

At first, we integrated an expression cassette which placed Cas9d under the transcriptional control of a strong promoter construct in the pel locus of RB128. The resulting strain was named BaC0303.

Strain BaC0303 (control) was then transformed with a plasmid containing an expression cassette designed to express a guide-RNA targeting either strand of the 5′ end of the yjbH coding sequence. The resulting transformants were named BaC0306 and BaC0307.

Strains BaC0303, BaC0306, and BaC0307 were grown in shake flasks (n=5) in LB medium and analysed for maltogenic amylase activity after 3 days.

The maltogenic amylase activity detected in supernatents obtained from strains BaC0306 and BaC0307 were doubled when compared to the control strain BaC0303 (see table below). Relatively, the doubling of activity was similar to results obtained from RB128 and an yjbH-mutated RB128 (yjbH: E159K) grown under the same conditions.

TABLE 5
Average (n = 5) maltogenic amylase
activity (MANU/ml) from shake flask cultures

| Strain | Avg MANU/ml (n = 5) | std dev |
|---|---|---|
| BaC0303 (control) | 4.76 | 0.35 |
| BaC0306 | 11.35 | 1.91 |
| BaC0307 | 10.12 | 0.82 |

1. A method for improving at least one property of a microorganism host cell by repressing the expression of one or more genome target sequence of interest, said method comprising the steps of:
a) providing a microorganism host cell comprising one or more genome target sequence of interest to be repressed, wherein each target sequence comprises or is flanked by a functional PAM sequence for a Class-II Cas9 enzyme;
b) transforming said host cell with:
i) a polynucleotide encoding a nuclease-null variant of the Class-II Cas9 enzyme,
ii) a polynuceotide encoding a single-guide RNA or a guide RNA complex for each target sequence to be repressed, said single-guide RNA or guide RNA complex comprising:
a) a first RNA sequence comprising 20 or more nucleotides that are at least 80% complementary to and capable of hybridizing to the at least one genome target sequence to be repressed and comprising a tracr mate sequence, and
b) a second RNA sequence comprising a tracr sequence complementary to and capable of hybridizing with the tracr mate sequence;
wherein the 20 or more nucleotides of the first RNA sequence hybridize with the one or more genome target sequence and wherein the nuclease-null variant Class-II Cas9 enzyme interacts with the single-guide RNA or the guide RNA complex and binds to the one or more genome target sequence, thereby repressing its expression and improving at least one property of said host cell.

2. The method of claim 1, wherein the microorganism host cell is a Bacillus host cell.

3. The method of claim 2, wherein the one or more genome target sequence of interest to be repressed comprises the mecA and/or the yjbH gene or homologues thereof.

4. The method of claim 1, wherein the one or more genome target sequence to be repressed comprises at least 20 nucleotides.

5. The method of claim 1, wherein the nuclease-null variant of the Class-II Cas9 enzyme comprises a substitution in the amino acid position corresponding to position 10 in the Streptomyces pyogenes Cas9 amino acid sequence.

6. The method of claim 1, wherein the nuclease-null variant of the Class-II Cas9 enzyme comprises a substitution in the amino acid position corresponding to position 840 in the Streptomyces pyogenes Cas9 amino acid sequence.

7. The method of claim 1, wherein the single-guide RNA or RNA complex comprises a first RNA comprising 20 or more nucleotides that are at least 90% complementary to and capable of hybridizing to the one or more genome target sequence.

8. The method of claim 1, wherein the microorganism host cell comprises a single-guide RNA comprising the first and second RNAs in the form of a single polynucleotide and wherein the tracr mate sequence and the tracr sequence form a stem-loop structure when hybridized with each other.

9. The method of claim 1, wherein two or more genome target sequences in the host cell are repressed.

10. The method of claim 1, wherein the improved property is an improved transformation efficiency.

11. The method of claim 1, wherein the improved property is a reduced protease expression.

12. The method of claim 1, wherein the improved property is an improved productivity or yield of a heterologous polypeptide produced by said host cell.

13. The method of claim 12, wherein the heterologous polypeptide is one or more enzyme selected from the group consisting of a hydrolase, isomerase, ligase, lyase, oxidoreductase, or a transferase.

14. The method of claim 1, wherein the one or more genome target sequence of interest is transiently repressed by a temperature-sensitive nuclease-null variant of the Class-II Cas9 enzyme.

15. A microorganism host cell having at least one improved property, wherein the expression of one or more genome target sequence of interest is repressed, said host cell comprising:
a) one or more repressed genome target sequence of interest, wherein each target sequence comprises or is flanked by a functional PAM sequence for a Class-II Cas9 enzyme;
b) a polynucleotide encoding a nuclease-null variant of the Class-II Cas9 enzyme,
c) a polynuceotide encoding a single-guide RNA or a guide RNA complex for each repressed target sequence, said single-guide RNA or guide RNA complex comprising:
i) a first RNA sequence comprising 20 or more nucleotides that are at least 80% complementary to and capable of hybridizing to the at least one repressed genome target sequence and comprising a tracr mate sequence, and
ii) a second RNA sequence comprising a tracr sequence complementary to and capable of hybridizing with the tracr mate sequence;
wherein the 20 or more nucleotides of the first RNA sequence hybridize with the one or more genome target sequence and wherein the nuclease-null variant Class-II Cas9 enzyme interacts with the single-guide RNA or the guide RNA complex and binds to the one or more genome target sequence, thereby repressing its expression and improving at least one property of said host cell.

16. The microorganism host cell of claim 15 which is a Bacillus host cell.

17. The microorganism host cell of claim 16, wherein the one or more genome target sequence of interest to be repressed comprises the mecA and/or the yjbH gene or homologues thereof.

18. The microorganism host cell of claim 15, wherein the one or more genome target sequence to be repressed comprises at least 20 nucleotides.

19. The microorganism host cell of claim 15, wherein the nuclease-null variant of the Class-II Cas9 enzyme comprises a substitution in the amino acid position corresponding to position 10 in the Streptomyces pyogenes Cas9 amino acid sequence.

20. The microorganism host cell of claim 15, wherein the nuclease-null variant of the Class-II Cas9 enzyme comprises a substitution in the amino acid position corresponding to position 840 in the Streptomyces pyogenes Cas9 amino acid sequence.

21. The microorganism host cell of claim 15, wherein the single-guide RNA or RNA complex comprises a first RNA comprising 20 or more nucleotides that are at least 90% complementary to and capable of hybridizing to the one or more genome target sequence.

22. The microorganism host cell of claim 15, wherein the microorganism host cell comprises a single-guide RNA comprising the first and second RNAs in the form of a single polynucleotide and wherein the tracr mate sequence and the tracr sequence form a stem-loop structure when hybridized with each other.

23. The microorganism host cell of claim 15, wherein two or more genome target sequences in the host cell are repressed.

24. The microorganism host cell of claim 15, wherein the improved property is an improved transformation efficiency.

25. The microorganism host cell of claim 15, wherein the improved property is a reduced protease expression.

26. The microorganism host cell of claim 15, wherein the improved property is an improved productivity or yield of a heterologous polypeptide produced by said host cell.

27. The method of claim 26, wherein the heterologous polypeptide is one or more enzyme selected from the group consisting of a hydrolase, isomerase, ligase, lyase, oxidoreductase, or a transferase.

28. The microorganism host cell of claim 15, wherein the one or more genome target sequence of interest is transiently repressed by a temperature-sensitive nuclease-null variant of the Class-II Cas9 enzyme.


SEQUENCE LISTING (43 sequences)

SEQ ID NO: 1 - DNA, 1144 bp, artificial sequence
  Table 1, fragment 1
ctctagaggatccccgggtaccgagctctgccttttagtccagctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatgagctcggaattcgtacgagttcctccacattcggagtatttctgaatgatagagccacacggtccacgttctcactggctaaccggatcaaatgatcttcaggagtcagcataatacatccagttcaggtagataagatttgaatttggtgacttgcttttgttcttcttctttcattttctgactaatccaaactggaaaaagcaggtcttttaacagattaggaggtttctgacatgcaccattcggtcactaaccgaatgcagtaaaggacactgtggtgcttgccagccattagggtattgaggaggtgatcaaaatgctaggtgacagtatttcgtcgaagtggacaagtcgtgaccaaatgacctcggatcgagggttggtcatggaggaaaaaattgatgtctggtgacaaagaggagtcatgatcatggcaccgccaacgagggaaaaaactcttcccgcatcgacacggtatgtgggcggtgacaaactaacttatagagtaaatttattagtcgaatgaaagacgcgctaaaaatgaggagggaagcga

SEQ ID NO: 2 - DNA, 4107 bp, artificial sequence
  Table 1, fragment 2
atggacaaaaaatacagcatcggcctggctattggcacaaattcagttggctgggcagttatcacagacgaatataaagttccgagcaaaaaatttaaagtcctgggcaatacagatcgccatagcatcaaaaaaaacctgattggcgcactgctgtttgattcaggcgaaacagcagaagcaacaagacttaaaagaacagcaagacgcagatatacaagacgcaaaaatcgcatttgctatctgcaagaaatctttagcaacgaaatggcgaaagtcgacgacagcttttttcatagactggaagaatcatttctggtcgaagaagataaaaaacacgaacgccatccgatttttggcaacattgttgatgaagtcgcgtatcatgaaaaatacccgacaatttatcatctgcgcaaaaaactggttgacagcacagataaagcagatcttcgcctgatttatctggcactggcacatatgatcaaatttagaggccattttctgatcgaaggcgatctgaatccggataattcagatgtcgacaaactgtttattcagctggtccagacatataaccagctgtttgaagaaaatccgattaatgcatcaggcgttgatgcaaaagcaattctgtcagcaagactgtcaaaatcaagacgcctggaaaatctgattgcacaactgcctggcgaaaaaaaaaatggactgtttggcaatcttattgcactgtcactgggcctgacaccgaactttaaatcaaattttgatctggcggaagatgcgaaactgcaactttcaaaagatacgtatgatgacgatctggataatctgctggcgcaaattggcgatcaatatgcagatctttttctggcagcgaaaaatctgtcagatgcaattctgctgtcagatattctgcgcgtcaatacagaaattacaaaagcaccgctgagcgcgagcatgattaaaagatatgatgaacatcatcaggacctgacactgctgaaagcactggttagacaacaactgccggaaaaatacaaagaaatcttttttgatcagagcaaaaacggctatgcaggctatattgatggcggagcatcacaagaagaattttacaaatttatcaaaccgatcctggaaaaaatggatggaacagaagaactgctggttaaactgaatcgcgaagatttactgagaaaacagcgcacatttgataatggctcaattccgcatcaaattcatctgggcgaactgcatgcgattcttagacgccaagaagatttttatccgtttctgaaagacaaccgggaaaaaattgaaaaaatcctgacatttcgcatcccgtattatgtcggaccgctggcaagaggcaattcaagatttgcatggatgacacgcaaaagcgaagaaacaattacaccgtggaattttgaagaagtcgttgataaaggcgcaagcgcacaatcatttattgaacgcatgacgaactttgacaaaaacctgccgaatgaaaaagtcctgccgaaacattcactgctgtatgaatactttacggtctataatgaactgacgaaagtcaaatatgtcacagaaggcatgagaaaaccggcatttctgtcaggcgaacagaaaaaagcgattgtcgatcttctgtttaaaacgaaccgcaaagtcacagtgaaacagctgaaagaagattactttaaaaaaatcgaatgctttgatagcgtcgaaatctcaggcgtcgaagatagatttaatgcaagcctgggcacatatcatgatctgctgaaaatcatcaaagataaagattttctggataacgaagaaaacgaagatatcctggaagatattgtgctgacactgacgctttttgaagatcgcgaaatgattgaagaacgcctgaaaacatatgcgcacctgtttgatgataaagtcatgaaacaacttaaacgcagacgctatacaggctggggcagactttcaagaaaactgattaacggcattcgcgataaacaaagcggcaaaacaatcctggattttctgaaatcagatggctttgcgaatcgcaattttatgcagctgattcatgatgacagcctgacgtttaaagaagatattcagaaagcacaagtttcaggccaaggcgattcactgcatgaacatattgcaaatctggcaggctcaccggcaatcaaaaaaggcattctgcaaacagttaaagtcgtcgatgaactggttaaagttatgggcagacataaaccggaaaacatcgttattgaaatggcacgcgaaaatcagacaacacaaaaaggacagaaaaattcacgcgaacggatgaaaagaattgaagaaggcattaaagaactgggcagccaaatcctgaaagaacatccggttgaaaatacacagctgcagaacgaaaaactgtatctgtattatctgcagaatggacgcgatatgtatgtcgatcaagaactggatattaatcgcctgagcgattatgatgtggatgctattgttccgcagagctttcttaaagatgatagcatcgataacaaagtcctgacacgctcagataaaaacagaggcaaatcagataatgtcccgtcagaagaggttgtcaaaaaaatgaaaaactactggcgtcaactgctgaacgcgaaacttattacacaacgcaaatttgacaatctgacaaaagcagaaagaggcggactgtcagaacttgataaagcgggttttatcaaaagacagctggtcgaaacacgccagattacaaaacatgttgcgcaaattctggatagccgcatgaacacaaaatatgacgaaaacgataaactgatccgggaagtcaaagtcattacgctgaaatcaaaactggtcagcgattttcgcaaagactttcagttttacaaagtccgcgaaatcaacaactaccatcatgcacatgatgcatatctgaatgcagttgtcggcacagcgcttatcaaaaaataccctaaactggaaagcgaatttgtctacggcgactataaagtctatgatgtccgcaaaatgattgcgaaaagcgaacaagaaattggcaaagcgacagcgaaatactttttttacagcaacatcatgaacttttttaaaacggaaatcacactggcgaacggcgaaattagaaaaagaccgcttattgaaacgaacggtgaaacaggcgaaattgtttgggataaaggcagagattttgcaacagttagaaaagttctgagcatgccgcaagtcaacatcgtgaaaaaaacagaagttcagacaggcggatttagcaaagaatcaattcttccgaaacgcaactcagacaaactgattgcgcgtaaaaaagactgggacccgaaaaaatacggtggctttgattcaccgacagttgcatattcagttctggttgttgcgaaagtggaaaaaggcaaatccaaaaaacttaaaagcgtgaaagaacttctgggcatcacaattatggaacgctcgagctttgaaaaaaacccgatcgactttctggaagccaaaggctataaagaagtgaaaaaagaccttattatcaaactgccgaaatacagcctgtttgaactggaaaatggcagaaaacgcatgctggcatcagcaggcgaacttcagaaaggcaatgaactggcactgccgtcaaaatatgttaactttctgtatctggcgagccattacgaaaaacttaaaggctcaccggaagataacgaacagaaacaactgtttgtcgaacagcataaacattacctggacgaaatcatcgaacaaatcagcgaattttcaaaacgcgttattctggcagatgcgaacctggataaagttcttagcgcatataacaaacaccgggataaaccgattagagaacaagcggaaaatatcattcacctgtttacactgacaaatcttggcgcaccggcagcgtttaaatactttgatacaacaattgaccgcaaacgctacacaagcacaaaagaagttctggacgcaacactgattcatcaatcaattacaggcctttatgaaacgagcattgatctgtcacaactgggaggcgattga

SEQ ID NO: 3 - DNA, 263 bp, artificial sequence
  Table 1, fragment 3
tgctgtccagactgtccgctgtgtaaaaaaaaggaataaaggggggttgacattattttactgatatgtataatataatttgtataagaaaatgtcttacaaagaagggaaggtgttttagagctagaaatagcaagttaaaataaggctagtccgttatcaacttgaaaaagtggcaccgagtcggtgctttgcggccgcagatctgggaccaataataatgactagagaagaaagaatgaagattgttcatgaaattaagg

SEQ ID NO: 4 - DNA, 1176 bp, artificial sequence
  Table 1, fragment 4
aacgaatattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactggctagcatggcaagacccagaaaagttctgggagatcccgctttgcataagcgtattatagtggatgacgcgggctttgttgtttacacttcttgcacctgctgacggcaatcatccctatctatgaaatcgagatttcagcaggccgttattttcgagagagttaaatctatattcattgtttttattttggtaaggacataccggattttaggtttggattaccggtcgagttagcttgtcttttcgcccactaccgtgtcgatgcgggagcaatttaccagaagcacttaccgattgatagttttttattccggtgattgcaaagtttcataaacaagcttgcatgcctgcagg

SEQ ID NO: 5 - DNA, 180 bp, artificial sequence
  Table 1, fragment 5
cagtttgaaaattatgtattatggagctcagaggaggagagtgagtaatgatgaggaaaaagagtttttggcttgggatgctgacggccttcatgctcgtgttcacgatggcattcagcgattccgcttctgctgctgaagaagcaaaagaaaaatatttaattggctttaatgagcagg

SEQ ID NO: 6 - DNA, 160 bp, artificial sequence
  Table 1, fragment 6
cttgtcaatgcagaagcggcaacgcgataacgcgtagggcccgcggctagcggccgcgtcgactagaagagcagagaggacggatttcctgaaggaaatccgtttttttattttgcccgtcttataaatttcgttgttcaataatcgcatccgattgcag

SEQ ID NO: 7 - DNA, 50 bp, artificial sequence
  Table 1, primer 1
ccgactgcgcaaaagacataatcgactctagaggatccccgggtaccgag

SEQ ID NO: 8 - DNA, 50 bp, artificial sequence
  Table 1, primer 2
cgatgctgtattttttgtccattcgcttccctcctcatttttagcgcgtc

SEQ ID NO: 9 - DNA, 24 bp, artificial sequence
  Table 1, primer 3
atggacaaaaaatacagcatcggc

SEQ ID NO: 10 - DNA, 47 bp, artificial sequence
  Table 1, primer 4
cacagcggacagtctggacagcatcaatcgcctcccagttgtgacag

SEQ ID NO: 11 - DNA, 23 bp, artificial sequence
  Table 1, primer 5
tgctgtccagactgtccgctgtg

SEQ ID NO: 12 - DNA, 49 bp, artificial sequence
  Table 1, primer 6
cccactttatccaatattcgttccttaatttcatgaacaatcttcattc

SEQ ID NO: 13 - DNA, 24 bp, artificial sequence
  Table 1, primer 7
ggaacgaatattggataaagtggg

SEQ ID NO: 14 - DNA, 50 bp, artificial sequence
  Table 1, primer 8
gtgtgcctattttttgtgaatcgacctgcaggcatgcaagcttgtttatg

SEQ ID NO: 15 - DNA, 25 bp, artificial sequence
  Table 1, primer 9
tcgattcacaaaaaataggcacacg

SEQ ID NO: 16 - DNA, 25 bp, artificial sequence
  Table 1, primer 10
gtcgattatgtcttttgcgcagtcg

SEQ ID NO: 17 - DNA, 23 bp, artificial sequence
  Table 1, primer 11
gtgcagttctcgtcgatgtccgg

SEQ ID NO: 18 - DNA, 25 bp, artificial sequence
  Table 1, primer 12
ggtcaagacattgatcaagcgcttc

SEQ ID NO: 19 - DNA, 50 bp, artificial sequence
  Table 1, primer 13
gaagcgcttgatcaatgtcttgaccgcaacgttcgcagatgctgctgaag

SEQ ID NO: 20 - DNA, 24 bp, artificial sequence
  Table 1, primer 14
ccataatacataattttcaaactg

SEQ ID NO: 21 - DNA, 31 bp, artificial sequence
  Table 1, primer 15
gaaaaatatttaattggctttaatgagcagg

SEQ ID NO: 22 - DNA, 26 bp, artificial sequence
  Table 1, primer 16
cgcgttgccgcttctgcattgacaag

SEQ ID NO: 23 - DNA, 25 bp, artificial sequence
  Table 1, primer 17
gttcaataatcgcatccgattgcag

SEQ ID NO: 24 - DNA, 24 bp, artificial sequence
  Table 1, primer 18
ctgcgcaaaagacataatcgattc

SEQ ID NO: 25 - DNA, 48 bp, artificial sequence
  Table 1, primer 19
gaatcgattatgtcttttgcgcagatgctggttctcgaccattcaagg

SEQ ID NO: 26 - DNA, 22 bp, artificial sequence
  Table 1, primer 20
cgtagacggcaagcccgtatcc

SEQ ID NO: 27 - DNA, 22 bp, artificial sequence
  Table 1, primer 21
ggtgaggaaatcgcgacagctg

SEQ ID NO: 28 - DNA, 23 bp, artificial sequence
  Table 1, primer 22
ccattgaagaaaacaagagcgtc

SEQ ID NO: 29 - DNA, 10421 bp, artificial sequence
  Table 1, plasmid pMOL3188 full sequence
gaattcgtacgagttcctccacattcggagtatttctgaatgatagagccacacggtccacgttctcactggctaaccggatcaaatgatcttcaggagtcagcataatacatccagttcaggtagataagatttgaatttggtgacttgcttttgttcttcttctttcattttctgactaatccaaactggaaaaagcaggtcttttaacagattaggaggtttctgacatgcaccattcggtcactaaccgaatgcagtaaaggacactgtggtgcttgccagccattagggtattgaggaggtgatcaaaatgctaggtgacagtatttcgtcgaagtggacaagtcgtgaccaaatgacctcggatcgagggttggtcatggaggaaaaaattgatgtctggtgacaaagaggagtcatgatcatggcaccgccaacgagggaaaaaactcttcccgcatcgacacggtatgtgggcggtgacaaactaacttatagagtaaatttattagtcgaatgaaagacgcgctaaaaatgaggagggaagcgaatggacaaaaaatacagcatcggcctggctattggcacaaattcagttggctgggcagttatcacagacgaatataaagttccgagcaaaaaatttaaagtcctgggcaatacagatcgccatagcatcaaaaaaaacctgattggcgcactgctgtttgattcaggcgaaacagcagaagcaacaagacttaaaagaacagcaagacgcagatatacaagacgcaaaaatcgcatttgctatctgcaagaaatctttagcaacgaaatggcgaaagtcgacgacagcttttttcatagactggaagaatcatttctggtcgaagaagataaaaaacacgaacgccatccgatttttggcaacattgttgatgaagtcgcgtatcatgaaaaatacccgacaatttatcatctgcgcaaaaaactggttgacagcacagataaagcagatcttcgcctgatttatctggcactggcacatatgatcaaatttagaggccattttctgatcgaaggcgatctgaatccggataattcagatgtcgacaaactgtttattcagctggtccagacatataaccagctgtttgaagaaaatccgattaatgcatcaggcgttgatgcaaaagcaattctgtcagcaagactgtcaaaatcaagacgcctggaaaatctgattgcacaactgcctggcgaaaaaaaaaatggactgtttggcaatcttattgcactgtcactgggcctgacaccgaactttaaatcaaattttgatctggcggaagatgcgaaactgcaactttcaaaagatacgtatgatgacgatctggataatctgctggcgcaaattggcgatcaatatgcagatctttttctggcagcgaaaaatctgtcagatgcaattctgctgtcagatattctgcgcgtcaatacagaaattacaaaagcaccgctgagcgcgagcatgattaaaagatatgatgaacatcatcaggacctgacactgctgaaagcactggttagacaacaactgccggaaaaatacaaagaaatcttttttgatcagagcaaaaacggctatgcaggctatattgatggcggagcatcacaagaagaattttacaaatttatcaaaccgatcctggaaaaaatggatggaacagaagaactgctggttaaactgaatcgcgaagatttactgagaaaacagcgcacatttgataatggctcaattccgcatcaaattcatctgggcgaactgcatgcgattcttagacgccaagaagatttttatccgtttctgaaagacaaccgggaaaaaattgaaaaaatcctgacatttcgcatcccgtattatgtcggaccgctggcaagaggcaattcaagatttgcatggatgacacgcaaaagcgaagaaacaattacaccgtggaattttgaagaagtcgttgataaaggcgcaagcgcacaatcatttattgaacgcatgacgaactttgacaaaaacctgccgaatgaaaaagtcctgccgaaacattcactgctgtatgaatactttacggtctataatgaactgacgaaagtcaaatatgtcacagaaggcatgagaaaaccggcatttctgtcaggcgaacagaaaaaagcgattgtcgatcttctgtttaaaacgaaccgcaaagtcacagtgaaacagctgaaagaagattactttaaaaaaatcgaatgctttgatagcgtcgaaatctcaggcgtcgaagatagatttaatgcaagcctgggcacatatcatgatctgctgaaaatcatcaaagataaagattttctggataacgaagaaaacgaagatatcctggaagatattgtgctgacactgacgctttttgaagatcgcgaaatgattgaagaacgcctgaaaacatatgcgcacctgtttgatgataaagtcatgaaacaacttaaacgcagacgctatacaggctggggcagactttcaagaaaactgattaacggcattcgcgataaacaaagcggcaaaacaatcctggattttctgaaatcagatggctttgcgaatcgcaattttatgcagctgattcatgatgacagcctgacgtttaaagaagatattcagaaagcacaagtttcaggccaaggcgattcactgcatgaacatattgcaaatctggcaggctcaccggcaatcaaaaaaggcattctgcaaacagttaaagtcgtcgatgaactggttaaagttatgggcagacataaaccggaaaacatcgttattgaaatggcacgcgaaaatcagacaacacaaaaaggacagaaaaattcacgcgaacggatgaaaagaattgaagaaggcattaaagaactgggcagccaaatcctgaaagaacatccggttgaaaatacacagctgcagaacgaaaaactgtatctgtattatctgcagaatggacgcgatatgtatgtcgatcaagaactggatattaatcgcctgagcgattatgatgtggatgctattgttccgcagagctttcttaaagatgatagcatcgataacaaagtcctgacacgctcagataaaaacagaggcaaatcagataatgtcccgtcagaagaggttgtcaaaaaaatgaaaaactactggcgtcaactgctgaacgcgaaacttattacacaacgcaaatttgacaatctgacaaaagcagaaagaggcggactgtcagaacttgataaagcgggttttatcaaaagacagctggtcgaaacacgccagattacaaaacatgttgcgcaaattctggatagccgcatgaacacaaaatatgacgaaaacgataaactgatccgggaagtcaaagtcattacgctgaaatcaaaactggtcagcgattttcgcaaagactttcagttttacaaagtccgcgaaatcaacaactaccatcatgcacatgatgcatatctgaatgcagttgtcggcacagcgcttatcaaaaaataccctaaactggaaagcgaatttgtctacggcgactataaagtctatgatgtccgcaaaatgattgcgaaaagcgaacaagaaattggcaaagcgacagcgaaatactttttttacagcaacatcatgaacttttttaaaacggaaatcacactggcgaacggcgaaattagaaaaagaccgcttattgaaacgaacggtgaaacaggcgaaattgtttgggataaaggcagagattttgcaacagttagaaaagttctgagcatgccgcaagtcaacatcgtgaaaaaaacagaagttcagacaggcggatttagcaaagaatcaattcttccgaaacgcaactcagacaaactgattgcgcgtaaaaaagactgggacccgaaaaaatacggtggctttgattcaccgacagttgcatattcagttctggttgttgcgaaagtggaaaaaggcaaatccaaaaaacttaaaagcgtgaaagaacttctgggcatcacaattatggaacgctcgagctttgaaaaaaacccgatcgactttctggaagccaaaggctataaagaagtgaaaaaagaccttattatcaaactgccgaaatacagcctgtttgaactggaaaatggcagaaaacgcatgctggcatcagcaggcgaacttcagaaaggcaatgaactggcactgccgtcaaaatatgttaactttctgtatctggcgagccattacgaaaaacttaaaggctcaccggaagataacgaacagaaacaactgtttgtcgaacagcataaacattacctggacgaaatcatcgaacaaatcagcgaattttcaaaacgcgttattctggcagatgcgaacctggataaagttcttagcgcatataacaaacaccgggataaaccgattagagaacaagcggaaaatatcattcacctgtttacactgacaaatcttggcgcaccggcagcgtttaaatactttgatacaacaattgaccgcaaacgctacacaagcacaaaagaagttctggacgcaacactgattcatcaatcaattacaggcctttatgaaacgagcattgatctgtcacaactgggaggcgattgatgctgtccagactgtccgctgtgtaaaaaaaaggaataaaggggggttgacattattttactgatatgtataatataatttgtataagaaaatgtcttacaaagaagggaaggtgttttagagctagaaatagcaagttaaaataaggctagtccgttatcaacttgaaaaagtggcaccgagtcggtgctttgcggccgcagatctgggaccaataataatgactagagaagaaagaatgaagattgttcatgaaattaaggaacgaatattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactggctagcatggcaagacccagaaaagttctgggagatcccgctttgcataagcgtattatagtggatgacgcgggctttgttgtttacacttcttgcacctgctgacggcaatcatccctatctatgaaatcgagatttcagcaggccgttattttcgagagagttaaatctatattcattgtttttattttggtaaggacataccggattttaggtttggattaccggtcgagttagcttgtcttttcgcccactaccgtgtcgatgcgggagcaatttaccagaagcacttaccgattgatagttttttattccggtgattgcaaagtttcataaacaagcttgcatgcctgcaggtcgattcacaaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacatatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcgatggatcagtacaagaaagatactgtatttcataaacaggaactgcaagaagttaaggatgagttacagaaggcaaataagcagttacagagtggaatagagcatatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagctagtacagtgactttatatcgtgctgcgagggcgaatttccctgggtttgagaaagggtttaataggcttaaagagaaattctttaatgattccaaatttgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggtttcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgactctagaggatccccgggtaccgagctctgccttttagtccagctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatgagctcg

SEQ ID NO: 30 - DNA, 4107 bp, artificial sequence; identical to SEQ ID NO: 2
  Table 1, codon-optimized Cas9d-encoding sequence

SEQ ID NO: 31 - DNA, 205 bp, artificial sequence
  A synthetic DNA fragment containing the scBAN promoter minus its ribosome binding site, plus the yjbH guide RNA targeting the DNA top strand.
aagctttgctgtccagactgtccgctgtgtaaaaaaaaggaataaaggggggttgacattattttactgatatgtataatataatttgtataagaaaatgtacaaacatctgggaaatacgttttagagctagaaatagcaagttaaaataaggctagtccgttatcaacttgaaaaagtggcaccgagtcggtgctttaagctt

SEQ ID NO: 32 - DNA, 44 bp, artificial sequence
  Primer 1216467
cctcgaggtcgacggtatcgataagctttgctgtccagactgtc

SEQ ID NO: 33 - DNA, 44 bp, artificial sequence
  Primer 1216468
gctgcaggaattcgatatcaagcttaaagcaccgactcggtgcc

SEQ ID NO: 34 - DNA, 205 bp, artificial sequence
  A synthetic DNA fragment containing the scBAN promoter minus its ribosome binding site, plus the yjbH guide RNA targeting the DNA bottom strand.
aagctttgctgtccagactgtccgctgtgtaaaaaaaaggaataaaggggggttgacattattttactgatatgtataatataatttgtataagaaaatgaccctttatgtcctgaatgcgttttagagctagaaatagcaagttaaaataaggctagtccgttatcaacttgaaaaagtggcaccgagtcggtgctttaagctt

SEQ ID NO: 35 - DNA, 18 bp, artificial sequence
  Primer 1209582
ctgcgtgtgcctacagat

SEQ ID NO: 36 - DNA, 51 bp, artificial sequence
  Primer 1216378
gcctattgagtatttcttatccattcggttccctcctcatttttatagagc

SEQ ID NO: 37 - DNA, 51 bp, artificial sequence
  Primer 1216377
gctctataaaaatgaggagggaaccgaatggataagaaatactcaataggc

SEQ ID NO: 38 - DNA, 52 bp, artificial sequence
  Primer 1217984
catcataatcacttaaacgattaatatctaattcttggtccacatacatgtc

SEQ ID NO: 39 - DNA, 1670 bp, artificial sequence
  A synthetic DNA template for the PCR amplification of a polynucleotide fragment comprising the S. pyogenes Cas9  (H840A) encoding portion of the cas9 gene.
tgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgaacgcgttaatcaataaaaaaacgctgtgcgg

SEQ ID NO: 40 - DNA, 20 bp, artificial sequence
  Primer 1213933
tgtggaccaagaattagata

SEQ ID NO: 41 - DNA, 25 bp, artificial sequence
  Primer 1214793
tttcttaatcaaagcagttccaacg

SEQ ID NO: 42 - DNA, 25 bp, artificial sequence
  Primer 1213934
cgttggaactgctttgattaagaaa

SEQ ID NO: 43 - DNA, 21 bp, artificial sequence
  Primer 1209587
gtgtcgatcagcttcttcagc